(19) United States
(12) Patent Application Publication
(10) Pub. No.: US 2025/0375211 A1
(43) Pub. Date: Dec. 11, 2025

(54) ASPIRATION LITHOTRIPSY DEVICE

(71) Applicant: Boston Scientific Scimed, Inc., Maple Grove, MN (US)

(72) Inventor: Cass Alexander Hanson, St. Paul, MN (US)

(73) Assignee: Boston Scientific Scimed, Inc., Maple Grove, MN (US)

(21) Appl. No.: 19/227,571

(22) Filed: Jun. 4, 2025

Related U.S. Application Data
(60) Provisional application No. 63/656,151, filed on Jun. 5, 2024.

(51) Int. Cl. A61B 17/22; A61B 17/00

(52) U.S. Cl. CPC A61B 17/22022; A61B 2017/00199; A61B 2017/00561; A61B 2017/22024; A61B 2217/005

(57) ABSTRACT

Medical devices and medical device systems are disclosed. An example medical device for treating a thrombotic lesion includes an elongate catheter including a proximal end region, a distal end region, an aspiration lumen and a wire lumen extending therein. The medical device also includes a support shaft coupled to the elongate catheter, wherein the support shaft includes a support lumen and a distal end coupled to a reflector. The medical device also includes an electrically conductive member extending within a portion of the wire lumen, and wherein the conductive member includes a distal end region positioned adjacent to the reflector. The medical device also includes an emitter coupled to the distal end region of the conductive member. Further, the emitter is configured to discharge an electrical pulse directed toward the distal end region of the elongate catheter.

### CROSS-REFERENCE TO RELATED APPLICATIONS

This application claims the benefit of priority under 35 U.S.C. § 119 of U.S. Provisional Application No. 63/656,151, filed Jun. 5, 2024, the entire disclosure of which is hereby incorporated by reference.

### TECHNICAL FIELD

The disclosure pertains to medical devices including an aspiration device coupled with a lithotripsy emitter. More particularly, the disclosure is directed to medical devices which may utilize lithotripsy in conjunction with aspiration to treat vessel occlusions.

### BACKGROUND

Many patients suffer from occluded vessels in the circulatory system which restricts blood flow. Arterial occlusions can be partial occlusions that reduce blood flow through the occluded portion of a blood vessel or total occlusions (e.g., chronic total occlusions) that substantially block blood flow through the occluded blood vessel. Occluded, stenotic, or narrowed blood vessels may be treated with a number of relatively non-invasive medical procedures including percutaneous transluminal angioplasty (PTA), percutaneous transluminal coronary angioplasty (PTCA), atherectomy, and lithotripsy. Additionally, venous occlusions such as DVT (Deep Vein Thrombosis) consist of thrombus at various levels of chronicity and can reduce blood flow to the point of total stasis. Aspirating or debulking thrombus is a common technique to restore an acceptable level of blood flow. Aspiration devices may be limited to generating zero atmosphere of pressure. In some instances, it may be beneficial to utilize lithotripsy in conjunction with aspiration to take advantage of an increase in pressure differential to debulk vascular occlusions and lesions. Of the known medical devices, systems, and methods, each has certain advantages and disadvantages. There is an ongoing need to provide alternative medical devices and systems, including devices and systems for treating thrombus occlusions or calcified lesions.

### SUMMARY

The disclosure is directed to several alternative designs, materials and methods of manufacturing medical device structures and assemblies. An example medical device for treating a thrombotic lesion includes an elongate catheter including a proximal end region, a distal end region, an aspiration lumen and a wire lumen extending therein. The medical device also includes a support shaft coupled to the elongate catheter, wherein the support shaft includes a support lumen and a distal end coupled to a reflector. The medical device also includes an electrically conductive member extending within a portion of the wire lumen, and wherein the conductive member includes a distal end region positioned adjacent to the reflector. The medical device also includes an emitter coupled to the distal end region of the conductive member. Further, the emitter is configured to discharge an electrical pulse directed toward the distal end region of the elongate catheter.

Alternatively, or additionally to any of the examples above, wherein the support lumen is in communication with the wire lumen, and wherein the electrically conductive member extends within at least a portion of the support lumen.

Alternatively, or additionally to any of the examples above, wherein the reflector is spaced away from a distal end of the elongate catheter.

Alternatively, or additionally to any of the examples above, wherein the reflector is parabolic-shaped and defines a concave surface facing the distal end of the elongate catheter.

Alternatively, or additionally to any of the examples above, wherein the emitter is configured to be positioned adjacent to the concave surface of the reflector.

Alternatively, or additionally to any of the examples above, wherein the electrical pulse creates a positive pressure which engages the concave surface and travels proximally toward the distal end of the elongate catheter.

Alternatively, or additionally to any of the examples above, wherein the proximal end region of the catheter is coupled to a pump, and wherein the pump is configured to generate a vacuum within the aspiration lumen of the catheter.

Alternatively, or additionally to any of the examples above, wherein the vacuum generated in the aspiration lumen of the catheter generates a negative pressure adjacent to the distal end of the catheter, and wherein the negative pressure is directed proximally into the aspiration lumen of the catheter.

Alternatively, or additionally to any of the examples above, wherein the positive pressure and the negative pressure are configured to transport thrombus from the thrombotic lesion into the aspiration lumen of the catheter.

Alternatively, or additionally to any of the examples above, wherein the pump is configured to maintain the vacuum within the lumen of the catheter to aspirate the thrombus from a patient.

Alternatively, or additionally to any of the examples above, wherein the positive pressure and the negative pressure are generated at substantially the same time period.

Alternatively, or additionally to any of the examples above, wherein the positive pressure and the negative pressure are generated in sequence.

Alternatively, or additionally to any of the examples above, wherein the distal end of the electrically conductive member is coupled to a controller, and wherein the controller is coupled to the console.

Alternatively, or additionally to any of the examples above, wherein the controller includes one or more actuators, and wherein the one or more actuators are configured to generate the electrical pulse, the vacuum within the catheter lumen, or both the electrical pulse and the vacuum within the catheter lumen.

Another example medical device for treating a thrombotic lesion includes an elongate catheter including a proximal end region, a distal end region, an aspiration lumen and a wire lumen extending therein. The medical device also includes a support shaft coupled to the elongate catheter, wherein the support shaft includes a support lumen and a distal end coupled to a first reflector and a second reflector. The medical device also includes an electrically conductive member extending within a portion of the wire lumen, wherein the conductive member includes a first end region positioned adjacent to the first reflector and a second end region positioned adjacent to the second reflector. The medical device also includes a first emitter coupled to the first end region of the conductive member, the first emitter positioned adjacent to the first reflector. The medical device also includes
a second emitter coupled to the second end region of the conductive member, the second emitter positioned adjacent to the second reflector. Further, the first emitter and the second emitter are configured to discharge an electrical pulse directed toward the distal end region of the elongate catheter.

Alternatively, or additionally to any of the examples above, wherein the support lumen is in communication with the wire lumen, and wherein the electrically conductive member extends within at least a portion of the support lumen.

Alternatively, or additionally to any of the examples above, wherein the first reflector is spaced away from a distal end of the elongate catheter, and wherein the second reflector is spaced away from the first reflector.

Alternatively, or additionally to any of the examples above, wherein the first emitter and the second emitter are each configured to discharge an electrical pulse at substantially the same time.

An example medical device system for treating a thrombotic lesion includes a console including a pump and an arc discharge generator and a controller coupled to the console. The system also includes an elongate catheter including a proximal end region, a distal end region, an aspiration lumen and a wire lumen extending therein, wherein the proximal end region coupled to the console. The system also includes a support shaft coupled to the elongate catheter, wherein the support shaft includes a support lumen and a distal end coupled to a reflector. The system also includes an electrically conductive member extending along the elongate catheter, wherein the conductive member includes a proximal end region and a distal end region positioned adjacent to the reflector, and wherein the proximal end region is coupled to the controller and an emitter coupled to the distal end region of the conductive member. Further, the emitter is configured to discharge an electrical pulse directed toward the distal end region of the elongate catheter.

Alternatively, or additionally to any of the examples above, wherein the electrical pulse creates a positive pressure which travels proximally toward the lumen of the catheter.

The above summary of some example embodiments is not intended to describe each disclosed embodiment or every implementation of the disclosure.

### BRIEF DESCRIPTION OF THE DRAWINGS

The disclosure may be more completely understood in consideration of the following detailed description of various embodiments in connection with the accompanying drawings, in which:

FIG. 1 illustrates a combination aspiration and lithotripsy medical device positioned in a body vessel of a patient;

FIG. 2 is a cross-sectional view of the combination aspiration and lithotripsy medical device taken along line 2-2 of FIG. 1;

FIG. 3 is a schematic block diagram of a console management system;

FIG. 4 illustrates the distal end region of a combination aspiration and lithotripsy medical device positioned in a body vessel of a patient;

FIG. 5 is a detailed view of a portion of the combination aspiration and lithotripsy medical device shown in FIG. 4;

FIG. 6 is a cross-sectional view of the combination aspiration and lithotripsy medical device taken along line 6-6 of FIG. 5;

FIG. 7 illustrates the lithotripsy emitter emitting energy;

FIG. 8 illustrates another combination aspiration and lithotripsy medical device. While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

Many patients suffer from occluded vessels in the circulatory system, and/or occluded ducts or other body lumens which may restrict bodily fluid (e.g., blood, bile, etc.) flow. Arterial occlusions can be partial occlusions that reduce blood flow through the occluded portion of a blood vessel or total occlusions (e.g., chronic total occlusions) that substantially block blood flow through the occluded blood vessel. Additionally, venous occlusions such as DVT (Deep Vein Thrombosis) may consist of thrombus at various levels of chronicity that can reduce blood flow to the point of total stasis. Revascularization techniques include using a variety of devices to debulk or remove an occlusion to create or enlarge an opening to restore blood flow in a region of the circulatory system. Additionally, blocked body lumens may be cleared to restore proper drainage.

In some cases, for example, ultrasound may be used to treat vascular lesions, such as fibrotic and calcified lesions, at various states of disease progression, ranging from soft plaques to severely calcified lesions. Vascular lesions that may lend themselves to being treated with ultrasound-based devices include irregular, severely calcified plaques that are located within and adjacent to vessel walls, and lesions that are more or less rigid and thus may be susceptible to being mechanically fatigued to failure. For example, sound-based devices may be used to produce standing wave pressure patterns within the thickness of the lesion, bending moments at the ends of the lesion, and/or resonance along the length of the lesion. In some cases, the high frequency mechanical action of ultrasound may also be effective in treating earlier state vascular lesions, including fibrotic and soft plaques. In some cases, an ultrasound device may apply a treatment of unfocused, near-field ultrasound waves to treat vascular lesions. While the devices or systems described herein are described with respect to vascular lesions and thrombosis, it should be understood that the devices or systems may be used in other applications, such as, but not limited to, peripheral calcified lesions, aortic valves, mitral valves, or non-vascular applications including the treatment of tumors. For example, the methods and systems described herein may be used in any conduit that includes or is adjacent to a target treatment site such as, but not limited to vascular lesions, peripheral lesions, tumors, etc.

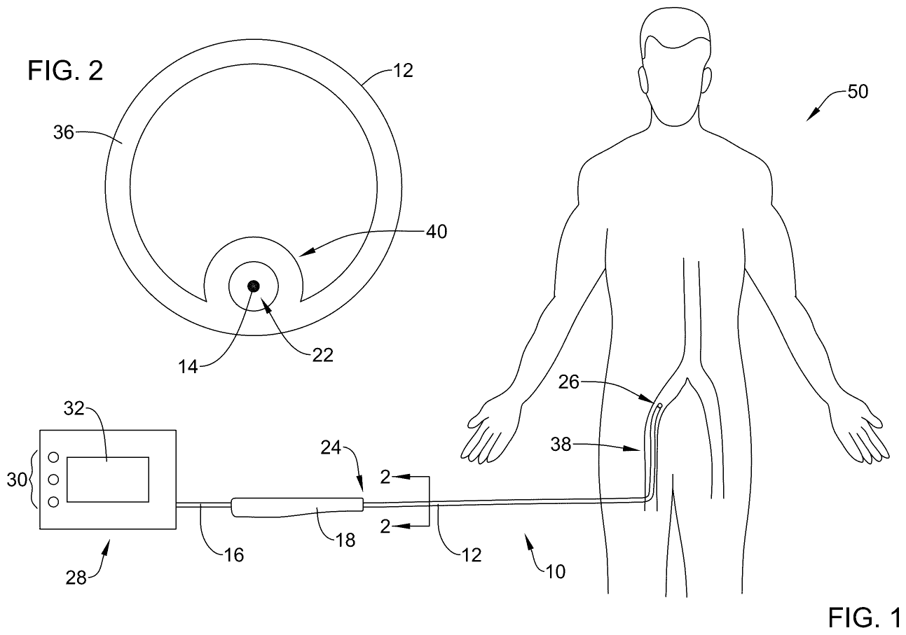

FIG. 1 illustrates a medical device 10 positioned within a patient 50. The medical device 10 disclosed herein may be utilized for temporary placement within a patient 50. FIG. 1 further illustrates that the medical device 10 may be inserted into and tracked through the patient's right femoral vein 38 (or artery) to a target treatment site (e.g., vascular thrombus, vascular lesion, vascular plaque, etc.). Among others, radial and pedal access sites are contemplated for the medical treatments discussed herein.

The medical device 10 may be a combination medical device which includes an aspiration device coupled with a lithotripsy emitter. For purposes of discussion herein, the medical device 10 may be referred to as a lithotripter assisted aspiration device. However, this is not intended to be limiting, as the medical device 10 may include additional components configured to treat a variety of medical conditions. For example, the medical device 10 may include or be used in conjunction with an embolic protection device, a balloon catheter, a thrombectomy device, an intravascular ultrasound device, an atherectomy device, etc. As will be discussed in greater detail herein, the medical device 10 may utilize lithotripsy in conjunction with aspiration to treat vessel occlusions.

FIG. 1 illustrates that the lithotripter assisted aspiration device 10 may include an elongated, tubular aspiration catheter 12 having a proximal end region 24 and a distal end region 26. The aspiration catheter 12 may include a medial region which extends between the proximal end region 24 and the distal end region 26. It is contemplated that the distal end region 26 may include a funnel shape. Further, the aspiration catheter 12 may include one or more lumens 22, 34 (shown in FIG. 2) which extend from the proximal end region 24 to the distal end region 26. As will be discussed in greater detail below, a console 28 may include a pump configured to generate a negative vacuum within the lumen of the aspiration catheter 12.

In some examples, the lithotripter assisted aspiration device 10 may include an elongated lithotripsy shaft 14 (e.g., electrically conductive wire) which extends within at least a portion of a lumen of the aspiration catheter 12 (e.g., the lithotripsy shaft 14 is shown positioned within the lumen 22 in FIG. 2).

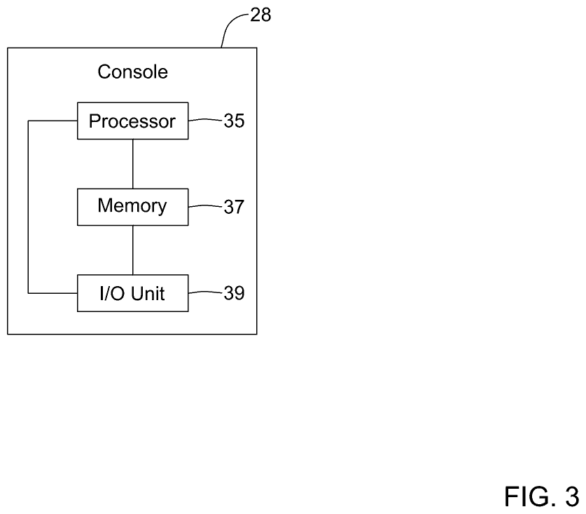

Additionally, it can be appreciated that the proximal end 24 of the aspiration catheter 12 and the proximal end of the lithotripsy shaft 14 may be attached to a controller 18. Further, FIG. 1 illustrates that the distal end region 26 of the lithotripsy shaft 14 may include a lithotripsy emitter 20 (shown in FIG. 3). FIG. 1 further illustrates that the controller 18 may be coupled to the console 28 via a connector shaft 16 (e.g., connector cable, connector catheter, etc.). It can be further appreciated that the connector shaft 16 may include an aspiration lumen which may be in communication with one or more lumens (e.g., lumen 34) of the aspiration catheter 12. Additionally, the connector shaft 16 may include a lithotripsy shaft or lithotripsy connector wire which is a separate element from the lithotripsy shaft 14 but which may be in communication with the lithotripsy shaft 14. Additionally, in other examples, the lithotripsy shaft 14 may extend from the console 18, through a lumen of the connector shaft 16, through the controller 18, through a lumen (e.g., lumen 22) of the aspiration catheter 12, to the distal end of the aspiration catheter 12.

It can be appreciated that the controller 18 may include one or more actuators (e.g., buttons, levers, dials, switches, etc.) designed to permit a user to control various functions of the lithotripter assisted aspiration device 10. For example, a user may be able to control the energy emitted by the lithotripsy emitter 20 (or plurality of lithotripsy emitters) via actuation of one or more actuators located on the controller 18. Additionally, a user may be able to control the vacuum through the aspiration catheter 12 via actuation of one or more actuators located on the controller 18.

Additionally, FIG. 1 illustrates that the console 28 may include one or more control knobs (e.g., buttons, knobs, dials, etc.) 30 and/or one or more displays 32. For example, FIG. 1 illustrates the console 28 may include a display 32. Additionally, while FIG. 1 illustrates the display 32 integrated into the console 28, it is contemplated that the display 32 may be a distinct component separate from the console 28. In other words, the display 32 may be a separate stand-alone display, apart from the console 28.

FIG. 2 is a cross-sectional view taken along line 2-2 of FIG. 1. As discussed herein, FIG. 2 illustrates the lithotripsy shaft 14 of the lithotripter assisted aspiration device 10 positioned within a lumen 22 of the aspiration catheter 12. It can be appreciated that the aspiration catheter 12 may be referred to as a multi-lumen catheter which may include a first lumen 34 configured for aspiration and a second lumen 22 configured to permit a lithotripsy shaft 14 to extend therein. It can be appreciated that the lumen 34 of the aspiration catheter 12 may be configured to pull vacuum through the aspiration catheter 12.

FIG. 3 illustrates that the console 28 may include, among other suitable components, one or more processors 35, memory 37, and an I/O unit 39. The processor 35 of the console 28 may include a single processor or more than one processor (e.g., a first processor 35 providing data/instructions to the display 32. The processor 35 may be configured to execute instructions, including instructions that may be loaded into the memory 37 and/or other suitable memory. Example processor components may include, but are not limited to, microprocessors, microcontrollers, multi-core processors, graphical processing units, digital signal processors, application specific integrated circuits (ASICs), field programmable gate arrays (FPGAs), discrete circuitry, and/or other suitable types of data processing devices. In some examples, the processor 35 of the console may be configured to execute program instructions. Program instructions may include, for example, firmware, microcode or application code that is executed by the processor 35, a microprocessor and/or microcontroller. The one or more processors 35 may be configured to each manage different functions. They may also be configured to concurrently perform the same functions (e.g., redundant system). Further yet, they may be configured such that a first processor 35 performs a given function and second processor 35 checks the result of the function of the first processor 35 for correctness (e.g., command-monitor system).

The memory 37 of the console 28 may include a single memory component or more than one memory component each working individually or with one another. Example types of memory may include random access memory (RAM), EEPROM, FLASH, suitable volatile storage devices, suitable non-volatile storage devices, persistent memory (e.g., read only memory (ROM), hard drive, optical disc memory, and/or other suitable persistent memory) and/or other suitable types of memory. The memory 37 may be or may include a non-transitory computer readable medium.

The I/O units 39 of the console 28 may include a single I/O component or more than one I/O component each working individually or with one another. Example I/O units 39 may be any type of communication port configured to communicate with other components of the lithotripter assisted aspiration device 10. Example types of I/O units 39 may include wired ports, wireless ports, radio frequency (RF) ports, Low-Energy Bluetooth ports, Bluetooth ports, Near-Field Communication (NFC) ports, HDMI ports, Wi-Fi ports, Ethernet ports, VGA ports, serial ports, parallel ports, component video ports, S-video ports, composite audio/video ports, DVI ports, USB ports, optical ports, and/or other suitable ports.

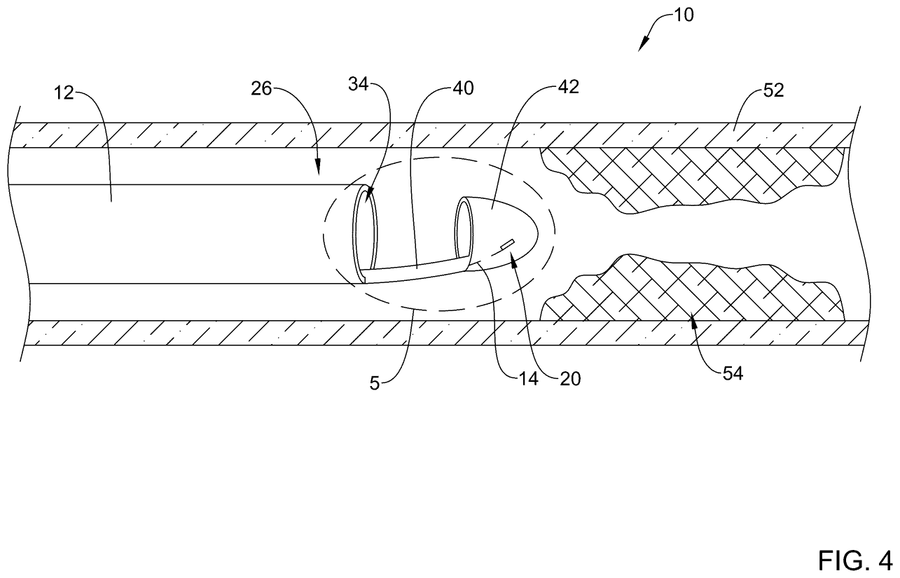

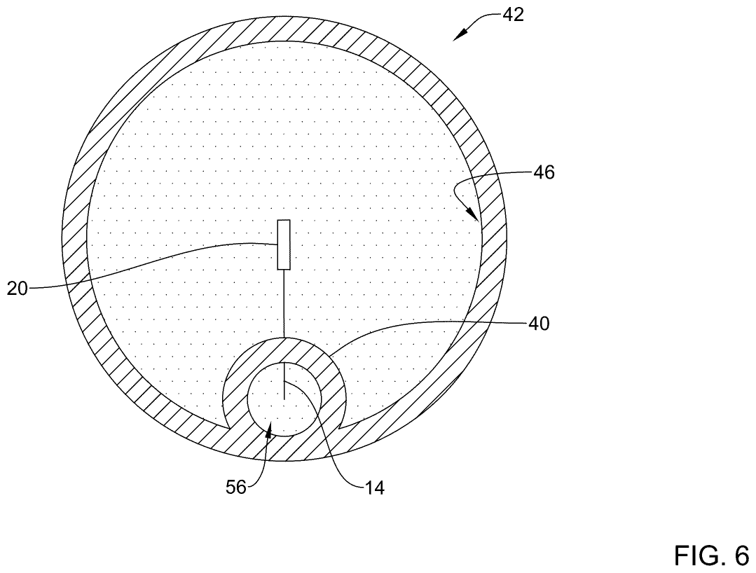

FIG. 4 illustrates a distal end portion of the lithotripter assisted aspiration device 10 positioned within a body vessel 52 and adjacent a target treatment site 54 (e.g., vascular thrombus, vascular lesion, vascular plaque, etc.). FIG. 4 illustrates that the lithotripter assisted aspiration device 10 may include a reflector support shaft 40 extending distally from a distal end of the aspiration catheter 12. It can be appreciated that the reflector support shaft 40 may be in fluid communication with the lumen 22 of the aspiration catheter 12. In other words, the reflector support shaft 40 may be an extension of the aspiration catheter 12 whereby the lumen 56 (shown in FIG. 6) of the reflector support shaft 40 may longitudinally align with the lumen 22 of the aspiration catheter 12.

Further, FIG. 4 further illustrates that a distal end of the reflector support shaft 40 may be attached to a reflector 42. In the example shown in FIG. 4, the reflector 42 may include a parabolic shape. However, this is not intended to be limiting, as the reflector 42 may include a variety of shapes including, but not limited to, a cone shape, a cube shape, a spherical shape, a half-sphere shape, a pyramid shape, a triangular-based pyramid shape, a cylinder shape, etc. The reflector 42 may include any shape that can provide directionality to a generated pressure wave. Additionally, it can be appreciated from FIG. 4 that the distal end of the reflector support shaft 40 may be attached to the proximal circumferential surface of the reflector 42, whereby the lumen 56 of the support shaft 40 may act as a conduit for the lithotripsy shaft 14 to extend through and exit the distal end of the aspiration catheter 12. It can be appreciated that the support shaft 40 may space the reflector 42 away from the distal end of the aspiration catheter 12.

In some examples, the reflector 42 may be formed from materials having high reflectivity properties (e.g., aluminum, silver, gold or similar materials). Additionally, materials that prevent heat distortion such as glass or ceramic may be incorporated into the construction of the reflector 42. Further, composite materials of carbon fiber may be incorporated into the construction of the reflector 42 to provide higher precision and efficiency. Further, any material that can provide directionality to a pressure wave may be contemplated for the construction of the reflector 42.

As discussed herein, FIG. 4 further illustrates a portion of the lithotripsy shaft 14 extending through the luminal space 56 (shown in FIG. 6) of support member 40. Further yet, FIG. 4 illustrates that the lithotripsy shaft 14 may extend into the reflector 42 such that the lithotripsy emitter 20 may be positioned within a portion of the reflector 42.

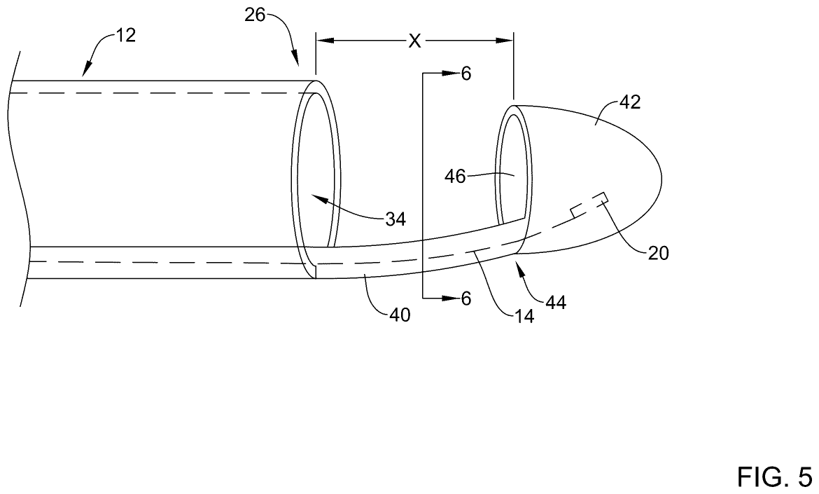

FIG. 5 illustrates the detailed view of FIG. 4. As discussed herein, FIG. 5 illustrates the distal end region 26 of the aspiration catheter 12. Further, FIG. 5 illustrates the reflector support shaft 40 extending distally from the distal end of the aspiration catheter 12. FIG. 5 further illustrates that the distal end of the reflector support shaft 40 may be attached to the proximalmost circumferential surface of the reflector 42 at attachment point 44, whereby the reflector support shaft 40 maintains a distal opening of the luminal space 56 (shown in FIG. 6). It can be appreciated that the lithotripsy shaft 14 may exit the reflector support shaft through this distal opening, whereby the lithotripsy emitter 20 may extend into and be positioned in the parabolic space of reflector 42.

Additionally, it can be appreciated that the support shaft 40 may space the reflector 42 away from the distal end of the aspiration catheter 12 a distance “X”. In some examples, the distance X may be about 1 mm to about 12 mm, or about 2 mm to about 11 mm, or about 3 mm to about 10 mm, or about 4 mm to about 9 mm, or about 5 mm to about 8 mm, or about 6 mm to about 7 mm, or about 5 mm. Additionally, it can be appreciated from FIGS. 4-5 that the inner surface of the reflector 42 may face toward the distal end region 26 of the aspiration catheter 12. In other words, the inner surface 46 of the reflector 42 may be defined as a concave surface which faces the distal end region 26 of the aspiration catheter 12. Additionally, while FIGS. 4-5 illustrate the reflector 42 spaced away from the distal end region of the aspiration catheter 12, it is further contemplated that, in other examples, the reflector 42 may be positioned within the lumen 34 of the distal end region 26 of the aspiration catheter 12.

FIG. 6 is a cross-sectional view taken along line 6-6 of FIG. 5. FIG. 6 illustrates a view of the reflector 42 as view from the distal end region 26 of the aspiration catheter 12. It can be appreciated that FIG. 6 illustrates the outer circumference of the proximal end of the reflector 42. As discussed herein, the reflector 42 illustrated in FIGS. 4-6 may include a parabolic shape, whereby FIG. 6 illustrates that the proximal end of the reflector 42 may include a circular, circumferential shape. It can be appreciated that a parabolic shape may be optimal to reflect a pressure wave, however, different shapes may reflect a pressure wave differently, and therefore are contemplated.

FIG. 6 further illustrates the lithotripsy shaft 14 extending within the lumen 56 of the support member 40. As discussed herein, the lumen 56 may permit the lithotripsy shaft 14 to extend distally into the interior of reflector 42. Further, as discussed herein, a lithotripsy emitter 20 may be attached to the distal end of the lithotripsy shaft 14 and may be positioned at the distalmost center region of the internal surface 46 of reflector 42.

As discussed herein, the lithotripsy support shaft 40 extends from the distal end of the aspiration catheter 12 and attaches to the inner surface of the reflector 42. However, in other examples, the reflector support shaft 40 may be a separate elongate shaft that extends within the lumen 34 of the aspiration catheter 12. In other words, in some examples, the aspiration catheter 12 may not be a multi-lumen catheter, but rather, it may include only a single lumen whereby the reflector support shaft 40 may be free-floating within the lumen 34 of the aspiration catheter 12. In this example, the reflector support shaft 40 may exit the distal end of the aspiration catheter 12 and then attach to the reflector 42 in a similar configuration as that described with respect to FIG. 4-5.

Additionally, in some examples, the lumen of the reflector support shaft 40 may permit a guidewire lumen to extend therein. It can be appreciated that examples in which the support shaft 40 includes a guidewire lumen may also include an aperture located in the reflector which is in communication with the guidewire lumen of the support shaft 40, thereby permitting a guidewire to extend within and through both the support shaft 40 and the wall of the reflector 42. All other guidewire configurations including monorail, over the wire, or hybrid designs are considered.

FIG. 6 further illustrates the lithotripsy emitter 20 positioned within the reflector 42. As discussed herein, the lithotripsy emitter 20 may be positioned along the distal end of the lithotripsy shaft 14. As illustrated in FIG. 6, the lithotripsy emitter may be positioned in a position which is in a substantially central region of the reflector 42. However, in other examples, the lithotripsy emitter may be positioned in other areas inside the reflector 42. For example, in some examples, the lithotripsy emitter 20 may be positioned adjacent to the inner surface 46 of the reflector 42. It can be appreciated that the lithotripsy emitter 20 may be positioned at any location between the reflector support shaft 40 and the inner surface 46 of the reflector 42.

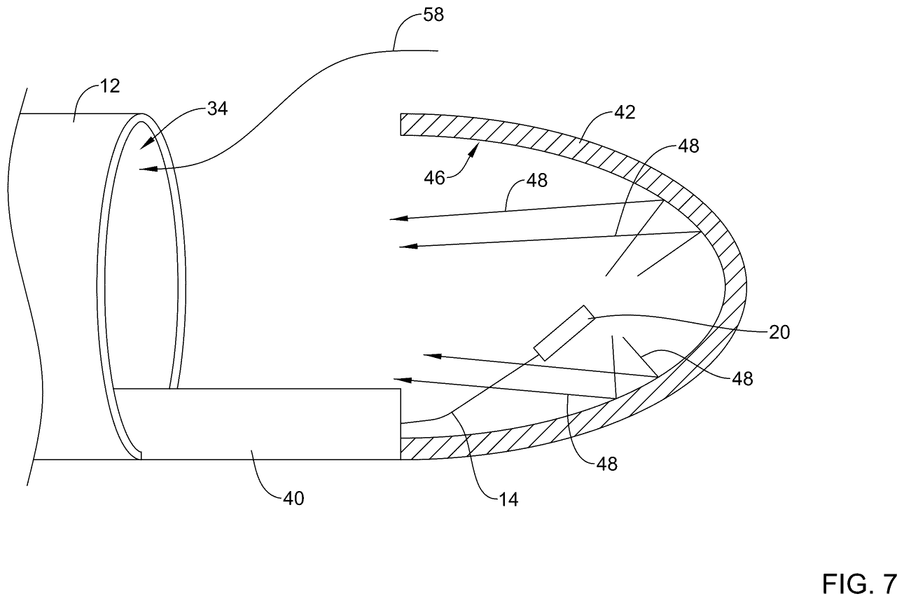

FIG. 7 illustrates the lithotripter assisted aspiration device 10 in which a user has powered (e.g., fired, etc.) the lithotripsy emitter 20 such the lithotripsy emitter 20 emits an electrical discharge 48 that may create sonic pressure waves that reflect off the inner surface 46 of the reflector 42 (e.g., a parabolic-shaped reflector 42) back toward the distal end region of the aspiration catheter 12. The lithotripsy emitter 20 may be configured to discharge electrical energy in rapid pulses, in spaced out interval pulses, at different power levels, or any combination thereof. As discussed herein, it can be appreciated that a user may power the lithotripsy emitter 20 via the one or more actuators (e.g., buttons, levers, dials, switches, etc.) positioned on the controller 18 (shown in FIG. 1) of the lithotripter assisted aspiration device 10. For example, a user may be able to control the amount of energy emitted by the lithotripsy emitter 20 (or plurality of lithotripsy emitters) via actuation of one or more actuators located on the controller 18.

It can be appreciated that, in some examples, the sonic pressure waves that reflect off the inner surface 46 of the reflector 42 may create a localized zone of positive pressure which is directed toward the distal end region 26 (including the lumen 34) of the aspiration catheter 12.

It can be further appreciated that in a close time period to (including coincident with) the powering of the lithotripsy emitter 20, a user may initiate a vacuum being pulled through the lumen 34 of the aspiration catheter 12. The flow of the vacuum being pulled into the lumen 34 of the aspiration catheter 12 is depicted by the directional arrows 58 in FIG. 7. Additionally, it can be appreciated that powering the aspiration catheter 12 to pull vacuum through the lumen 34 may create a localized zone of negative pressure adjacent to the distal end region 26 (e.g., the distal opening) of the aspiration catheter 12.

As discussed herein, it can be appreciated that a user may power the vacuum of the aspiration catheter 12 via the one or more actuators (e.g., buttons, levers, dials, switches, etc.) positioned on the controller 18 (shown in FIG. 1) of the lithotripter assisted aspiration device 10. Additionally, in some examples, it can be appreciated that the console 28 may include a vacuum source (e.g., a pump to pull vacuum through the lumen 34 of the aspiration catheter 12), an arc discharge generator (to generate an electrical discharge within the lithotripsy emitter 20), or both a vacuum source and an arc discharge generator. Further, in some examples, the controller 18 may include an actuator (e.g., switch, button, etc.) which is configured to initiate (e.g., power) a vacuum being pulled through the lumen 34 of the aspiration catheter 12. It can be appreciated that the same actuator that is used to power the vacuum being pulled through the lumen 34 of the aspiration catheter 12 may also power the lithotripsy emitter 20 to generate an electrical discharge from the lithotripsy emitter 20.

Referring to FIG. 4, it can be appreciated that when used in a medical procedure, a user may advance the distal end region of the lithotripter assisted aspiration device 10 to a position in which the reflector 42 and the lithotripsy emitter 20 are positioned adjacent to or nested within a target treatment site 54 (e.g., vascular thrombus, vascular lesion, vascular plaque, etc.). It can be further appreciated that when positioned adjacent to or nested within a target treatment site 54, a user may actuate an actuator (e.g., switch, button, etc.) on the controller 18 which ignites the lithotripsy emitter 20, thereby creating a sonic pressure wave directed toward the distal end region (e.g., the distal opening) of the aspiration catheter 12. The sonic pressure wave created by the electrical discharge from the emitter 20 may break apart a portion or all of the vascular thrombus present at the target tissue site 54 and direct it toward the distal end region 26 (e.g., the distal opening) of the aspiration catheter 12. Further, as discussed herein, it can be appreciated that powering the aspiration catheter 12 to pull vacuum through the lumen 34 may create a localized zone of negative pressure adjacent to the distal end region 26 (e.g., the distal opening) of the aspiration catheter 12. Accordingly, it can be appreciated that the zone of positive pressure (e.g., proximally-directed pressure wave) generated by the lithotripsy emitter 20 may be generated coincident with the negative pressure wave generated by the aspiration catheter 12 to direct the fragments of broken up thrombus of the target tissue site 54 into the lumen 34 of the aspiration catheter 12 whereby it is aspirated out of the patient. In other words, the positive pressure (e.g., proximally-directed pressure wave) generated by the lithotripsy emitter 20 may push broken up thrombus toward the lumen 34 of the aspiration catheter 12 while the negative pressure created by the powering of the vacuum of the aspiration catheter 12 may pull the broken thrombus toward the lumen 34 of the aspiration catheter 12.

It can be further appreciated that, in some instances, aspiration catheters can get plugged with thrombus and may be prevented from aspirating the thrombus out of the catheter. Accordingly, in some examples, it is contemplated that the lithotripter assisted aspiration device 10 may not include a reflector 42 nor a support shaft 40. In such a configuration, the lithotripsy emitter 20 may be attached to the distal end of lithotripsy shaft 14 and be positioned within the distal end of the lumen 34. The purpose of the lithotripsy emitter 20 is to assist in unplugging and macerating thrombus or lesion material. In this design, the lithotripsy emitter 20 may be fired separately, randomly, in concert with, or in sync with the negative pressure generated within the lumen 34 of aspiration catheter 12.

Additionally, it is contemplated that any of the lithotripter assisted aspiration devices disclosed herein may be configured such that the lithotripsy shaft 14 may be embedded within and/or extend through the wall of the aspiration catheter 12. Alternatively, it is contemplated that any of the lithotripter assisted aspiration devices disclosed herein may be configured such that the lithotripsy shaft 14 may free float in the aspiration lumen 34 of the aspiration catheter 12.

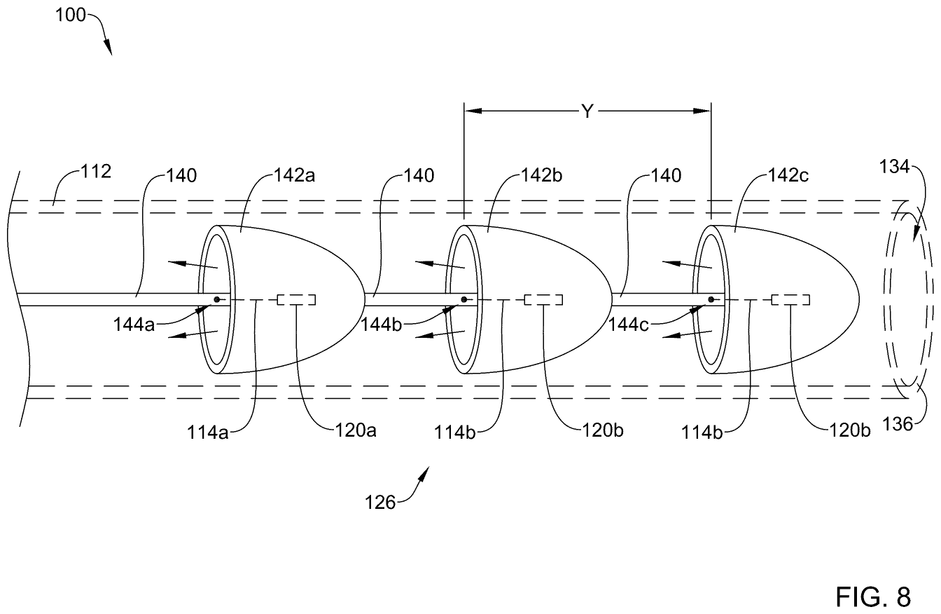

FIG. 8 illustrates another example lithotripter assisted aspiration device 100. The lithotripter assisted aspiration device 100 may be similar in form and function to the lithotripter assisted aspiration device 10 described herein. For example, FIG. 8 illustrates the distal end region 126 of an aspiration catheter 112. It can be appreciated that the aspiration catheter 112 may be coupled to the console 28 (shown in FIG. 1) and operate similarly to the aspiration catheter 12 described herein.

FIG. 8 further illustrates that the lithotripter assisted aspiration device 100 may include a plurality of reflectors 142a, 142b, 142c attached to a support shaft 140. The support shaft 140 may extend through the reflectors 142a, 142b whereby the distal end of the support shaft 140 may be attached to the reflector 142c. It can be appreciated that each of the reflectors 142a, 142b, 142c and the support shaft 140 may be similar in form and function to the reflector 42 and the support shaft 40 described herein. FIG. 8 further illustrates that each of the reflectors 142a, 142b, 142c may be aligned with one another along the longitudinal axis of the support shaft 140. Additionally, FIG. 8 illustrates that each of the reflectors 142a, 142b, 142c may be positioned within a lumen 134 of the aspiration catheter 112.

FIG. 8 further illustrates that the lithotripter assisted aspiration device 100 may include a lithotripsy emitter 120a positioned within the reflector 142a, a lithotripsy emitter 120b positioned within the reflector 142b and a lithotripsy emitter 120c positioned within the reflector 142c. It can be appreciated that each of the lithotripsy emitters 120a, 120b, 120c may be similar in form and function to the lithotripsy emitter 20 described herein.

Additionally, FIG. 8 illustrates that the reflectors 142a, 142b, 142c may be spaced away from one another along the longitudinal axis of the support shaft 140 a distance “Y”. In some examples, the distance Y may be about 1 mm to about 30 mm, or about 2 mm to about 29 mm, or about 3 mm to about 28 mm, or about 4 mm to about 27 mm, or about 5 mm to about 26 mm, or about 6 mm to about 25 mm, or about 7 mm to about 24 mm, or about 8 mm to about 23 mm, or about 9 mm to about 22 mm, or about 10 mm to about 21 mm, or about 11 mm to about 20 mm, or about 12 mm to about 19 mm, or about 13 mm to about 18 mm, or about 14 mm to about 17 mm, or about 15 mm to about 16 mm. However, while FIG. 8 illustrates that reflectors 142a, 124b, 142c are spaced equidistant apart from one another, it can be appreciated that in other examples, one or more of the reflectors 142a, 142b, 142c may be unequally spaced away from one or more of the reflectors 142a, 142b, 142c along the support shaft 140.

Like that described above with respect to the lithotripter assisted aspiration device 10, it can be appreciated that when positioned adjacent to or nested within a target treatment site 54 (shown in FIG. 4), a user may actuate an actuator (e.g., switch, button, etc.) on the controller 18 which ignites each of the lithotripsy emitters 120a, 120b, 120c, thereby creating one or more sonic pressure waves directed proximal to the distal end region of 126 (e.g., the distal opening) of the aspiration catheter 112. The one or more sonic pressure waves created by the electrical discharge from the emitters 120a, 120b, 120c may break apart a portion or all of the vascular thrombus present at the target tissue site 54 (shown in FIG. 4) and direct it proximally within the lumen 134 of the aspiration catheter 112. It can be further appreciated that each of the emitters 120a, 120b, 120c may be ignited at the same time or in any sequence. For example, each of the emitters 120a, 120b, 120c may be ignited at the same time. Alternatively, the emitter 120a may be ignited first, followed by the emitter 120b, followed by the emitter 120c. Alternatively, the emitter 120c may be ignited first, followed by the emitter 120b, followed by the emitter 120a. Alternatively, the emitter 120a may be ignited first, followed by the emitter 120c, followed by the emitter 120b. Alternatively, the emitter 120b may be ignited first, followed by the emitter 120a, followed by the emitter 120c. Alternatively, the emitter 120b may be ignited first, followed by the emitter 120c, followed by the emitter 120a. Alternatively, the emitter 120c may be ignited first, followed by the emitter 120a, followed by the emitter 120b. Additionally, any plurality of the emitters 120a, 120b, 120c may be ignited at the same time followed by the ignition of the remaining emitter.

Like that described above with respect to the lithotripter assisted aspiration device 10, each of the lithotripsy emitters 120a, 120b, 120c of the lithotripter assisted aspiration device 100 may each create a localized zone of positive pressure, the positive pressure of which is directed proximally within the lumen 134 of the aspiration catheter 112. Additionally, the lithotripsy emitters 120a, 120b, 120c may be configured to discharge electrical energy in rapid pulses, in spaced out interval pulses, at different power levels, or any combination thereof. As discussed herein, it can be appreciated that a user may power the lithotripsy emitters 120a, 120b, 120c via the one or more actuators (e.g., buttons, levers, dials, switches, etc.) positioned on the controller 18 (shown in FIG. 1) of the lithotripter assisted aspiration device 100. For example, a user may be able to control the amount of energy emitted by the lithotripsy emitters 120a, 120b, 120c via actuation of one or more actuators located on the controller 18.

Further, it can be appreciated that powering the aspiration catheter 112 to pull vacuum through the lumen 134 coincident with powering the lithotripsy emitters 120a, 120b, 120c may create one or more localized zones of negative pressure within the distal end region 126 (e.g., the distal opening) of the aspiration catheter 112. Further, as discussed herein, it can be appreciated that powering the aspiration catheter 112 to pull vacuum through the lumen 134 may create a localized zone of negative pressure within the distal end region 126 (e.g., the distal opening) of the aspiration catheter 112. Accordingly, it can be appreciated that the zones of positive pressure (e.g., a proximally-directed pressure wave) generated by the lithotripsy emitters 120a, 120b, 120c may be generated coincident with a vacuum-generated zone of negative pressure generated within the aspiration catheter 112 to direct the fragments of broken up thrombus of the target tissue site 54 (shown in FIG. 1) into the lumen 134 of the aspiration catheter 112, whereby it is aspirated out of the patient. In other words, the positive pressure (e.g., proximally-directed pressure wave) generated by the lithotripsy emitters 120a, 120b, 120c may push broken up thrombus toward the lumen 134 of the aspiration catheter 112 while the negative pressure created by the powering of the vacuum of the aspiration catheter 112 may pull the broken thrombus toward the lumen 134 of the aspiration catheter 112.

Additionally, FIG. 8 illustrates that a lithotripsy shaft (similar to the lithotripsy shaft 14 described herein) may extend through a lumen of the support shaft 140, whereby a first portion 114a of the lithotripsy shaft may exit the support shaft 140 at a first exit point 144a, a second portion 114b of the lithotripsy shaft may exit the support shaft 140 at a second exit point 144b, and a third portion 114c of the lithotripsy shaft may exit the support shaft 140 at a third exit point 144c. Further, it can be appreciated that each of the first portion 114a, second portion 114b and third portion 114c of the lithotripsy shaft may include a lithotripsy emitter 120a, 120b, 120c attached thereto, respectively. It can be appreciated from FIG. 8 that the first lithotripsy emitter 120a may be positioned within the first reflector 142a, the second lithotripsy emitter 120b may be positioned within the second reflector 142b, and the third lithotripsy emitter 120c may be positioned within the third reflector 142c. As illustrated in FIG. 8 and described herein, the lithotripsy emitters 120a, 120b, 120c may be deposed in the central region of their respective reflector 142a, 142b, 142c or may be positioned adjacent to any of the available interior space of the inner reflector surface. In yet other examples, the functional components of the lithotripsy device and the aspiration device may be separate, distinct components from one another.

In some embodiments, the medical devices 10, 100 and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), expanded polytetrafluoroethylene (ePTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments components can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-NR and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, the medical devices 10, 100 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility may be imparted into the medical devices 10, 100 disclosed herein. For example, the medical devices 10, 100 may be made of a material that does not substantially distort the image and create substantial artifacts (i.e., gaps in the image). Certain ferromagnetic materials, for example, may not be suitable because they may create artifacts in an MRI image. The system, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-NR and the like), nitinol, and the like, and others.

In some embodiments, the medical devices 10, 100 disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antincoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the present disclosure. This may include, to the extent that it is appropriate, the use of any of the features of one example embodiment being used in other embodiments. The scope of the present disclosure is, of course, defined in the language in which the appended claims are expressed.

What is claimed is:

1. A medical device for treating a thrombotic lesion, comprising:
an elongate catheter including a proximal end region, a distal end region, an aspiration lumen and a wire lumen extending therein;
a support shaft coupled to the elongate catheter, wherein the support shaft includes a support lumen and a distal end coupled to a reflector;
an electrically conductive member extending within a portion of the wire lumen, and wherein the conductive member includes a distal end region positioned adjacent to the reflector;
an emitter coupled to the distal end region of the conductive member;
wherein the emitter is configured to discharge an electrical pulse directed toward the distal end region of the elongate catheter.

2. The medical device of claim 1, wherein the support lumen is in communication with the wire lumen, and wherein the electrically conductive member extends within at least a portion of the support lumen.

3. The medical device of claim 1, wherein the reflector is spaced away from a distal end of the elongate catheter.

4. The medical device of claim 3, wherein the reflector is parabolic-shaped and defines a concave surface facing the distal end of the elongate catheter.

5. The medical device of claim 4, wherein the emitter is configured to be positioned adjacent to the concave surface of the reflector.

6. The medical device of claim 5, wherein the electrical pulse creates a positive pressure which engages the concave surface and travels proximally toward the distal end of the elongate catheter.

7. The medical device of claim 6, wherein the proximal end region of the catheter is coupled to a pump, and wherein the pump is configured to generate a vacuum within the aspiration lumen of the catheter.

8. The medical device of claim 7, wherein the vacuum generated in the aspiration lumen of the catheter generates a negative pressure adjacent to the distal end of the catheter, and wherein the negative pressure is directed proximally into the aspiration lumen of the catheter.

9. The medical device of claim 8, wherein the positive pressure and the negative pressure are configured to transport thrombus from the thrombotic lesion into the aspiration lumen of the catheter.

10. The medical device of claim 9, wherein the pump is configured to maintain the vacuum within the lumen of the catheter to aspirate the thrombus from a patient.

11. The medical device of claim 10, wherein the positive pressure and the negative pressure are generated at substantially the same time period.

12. The medical device of claim 10, wherein the positive pressure and the negative pressure are generated in sequence.

13. The medical device of claim 11, wherein the distal end of the electrically conductive member is coupled to a controller, and wherein the controller is coupled to the console.

14. The medical device of claim 13, wherein the controller includes one or more actuators, and wherein the one or more actuators are configured to generate the electrical pulse, the vacuum within the catheter lumen, or both the electrical pulse and the vacuum within the catheter lumen.

15. A medical device for treating a thrombotic lesion, comprising:
an elongate catheter including a proximal end region, a distal end region, an aspiration lumen and a wire lumen extending therein;
a support shaft coupled to the elongate catheter, wherein the support shaft includes a support lumen and a distal end coupled to a first reflector and a second reflector;
an electrically conductive member extending within a portion of the wire lumen, wherein the conductive member includes a first end region positioned adjacent to the first reflector and a second end region positioned adjacent to the second reflector;
a first emitter coupled to the first end region of the conductive member, the first emitter positioned adjacent to the first reflector;
a second emitter coupled to the second end region of the conductive member, the second emitter positioned adjacent to the second reflector;
wherein the first emitter and the second emitter are configured to discharge an electrical pulse directed toward the distal end region of the elongate catheter.

16. The medical device of claim 15, wherein the support lumen is in communication with the wire lumen, and wherein the electrically conductive member extends within at least a portion of the support lumen.

17. The medical device of claim 16, wherein the first reflector is spaced away from a distal end of the elongate catheter, and wherein the second reflector is spaced away from the first reflector.

18. The medical device of claim 17, wherein the first emitter and the second emitter are each configured to discharge an electrical pulse at substantially the same time.

19. A medical device system for treating a thrombotic lesion, comprising:
a console including a pump and an arc discharge generator;
a controller coupled to the console;
an elongate catheter including a proximal end region, a distal end region, an aspiration lumen and a wire lumen extending therein, wherein the proximal end region coupled to the console;
a support shaft coupled to the elongate catheter, wherein the support shaft includes a support lumen and a distal end coupled to a reflector;
an electrically conductive member extending along the elongate catheter, wherein the conductive member includes a proximal end region and a distal end region positioned adjacent to the reflector, and wherein the proximal end region is coupled to the controller;
an emitter coupled to the distal end region of the conductive member;
wherein the emitter is configured to discharge an electrical pulse directed toward the distal end region of the elongate catheter.

20. The medical device system of claim 19, wherein the electrical pulse creates a positive pressure which travels proximally toward the lumen of the catheter.
